(19) United States
(12) Patent Application Publication
(10) Pub. No.: US 2021/0005322 A1
(43) Pub. Date: Jan. 7, 2021

(54) BIOLOGICAL FLUID ANALYSIS AND PERSONALIZED HYDRATION ASSESSMENT SYSTEMS

(71) Applicant: MX3 Diagnostics, Inc., Austin, TX (US)

(72) Inventors: Duc Hau Huynh, Lalor (AU); Michael Erlichster, Caulfield North (AU); Thanh Cong Nguyen, Sunshine West (AU); Duc Phuong Nguyen, Deer Park (AU); Efstratios Skafidas, Thornbury (AU); Hsien Ming, Footscray (AU); Gursharan Chana, Fitroy North (AU); Ting Ting Lee, Footscray (AU); Chathurika Darshani Abeyrathne, Mitcham (AU); You Liang, Carlton (AU); Trevor John Kilpatrick, Parkville (AU); Michael Luther, Austin, TX (US); Alan Dayvault Luther, Edina, MN (US)

(21) Appl. No.: 16/918,150

(22) Filed: Jul. 1, 2020

Related U.S. Application Data
(60) Provisional application No. 62/957,527, filed on Jan. 6, 2020.
(60) Provisional application No. 62/876,263, filed on Jul. 19, 2019.
(60) Provisional application No. 62/869,210, filed on Jul. 1, 2019.

(51) Int. Cl. G16H 50/30; G16H 10/60; G16H 10/40; G16H 50/70; G01N 33/50; B01L 3/00

(52) U.S. Cl. CPC G16H 50/30; G16H 10/60; G16H 10/40; B01L 2300/0825; G01N 33/50; B01L 3/502715; G16H 50/70

(57) ABSTRACT

A method of measuring an analyte in a bodily fluid sample and combining measurement data from multiple users may involve initiating a wireless connection between a handheld analyzer and a smart computing device on which an analyte analysis application has been downloaded and inserting a test strip into the handheld analyzer. The method may further involve collecting a sample of a bodily fluid on the test strip, measuring, with the handheld analyzer, a concentration of at least one analyte in the sample, wirelessly communicating the measured concentration from the handheld analyzer to the smart computing device, and displaying the measured concentration on the smart computing device. Finally, the method may involve transmitting the measured concentration to a database and organizing data including the measured concentration and at least one additional measured analyte concentration from at least one additional user on the database.

### CROSS REFERENCE TO RELATED APPLICATIONS

This application claims priority to U.S. Provisional patent application Ser. No. 62/869,210, titled “Biological Fluid Analysis System,” filed Jul. 1, 2019; 62/876,263, titled “Personalized Hydration Assessment and Fluid Replenishment,” filed Jul. 19, 2020; and 62/957,527, titled “Personalized Hydration Assessment and Fluid Replenishment,” filed Jan. 6, 2020. The full disclosures of all the above-referenced provisional patent applications are hereby incorporated by reference herein.

### TECHNICAL FIELD

This application describes biomedical systems and methods. More specifically, the application describes systems and methods for analyzing biological fluids, such as saliva and sweat, assessing water and electrolyte loss in a human subject using one or more biomarkers, and recommending a fluid replenishment protocol.

### BACKGROUND

Sweating (or “perspiration”) is a technique the human body uses to regulate its temperature. During periods of physical exertion and/or environmental heat stress, sweat is excreted by the skin, which results in cooling via evaporation on the skin surface. While sweat is made up of approximately 99% water, sweat also typically contains other compounds, such as metabolites and ions. The excretion rate and composition of sweat are intrinsic to an individual and subject to biological variation. Excretion rate and sweat composition can also differ greatly, based on age, diet, activity, current fitness level and environmental conditions.

Appropriate hydration in the human body is vital for health and proper functioning of the body organs. Water is lost from the body during respiration, perspiration and urination. Decrease in body mass related to fluid loss of just a few percent can negatively impact cardiovascular function, thermal dissipation, and exercise performance. Dehydration can cause headaches, light-headedness, dizziness, fainting, and in extreme cases delirium, unconsciousness and even death. Large losses of body mass (e.g., greater than 5%) may result in heat exhaustion, heat stroke, loss of consciousness, organ damage and even death. Additionally, the loss of ions, predominantly sodium, through perspiration can result in fatigue and muscle cramping if not effectively replaced though ingested fluids. As there is a large amount of inter- and intra-individual variability in the volume and composition of sweat loss, fluid replacement strategies should ideally be individually tailored, to ensure both water and sodium losses are replenished to minimize the detrimental effects of physical exertion and/or heat stress on health and performance. Hyponatremia (“over-hydration”) can also detrimentally affect the body's functioning, particularly during exercising, and can even lead to death in extreme cases.

Dehydration is an excessive loss of body fluid. In physiological terms, dehydration may entail a deficiency of fluid within an organism. Dehydration can be caused by losing too much fluid, not drinking enough fluid, or both. Vomiting, diarrhea, and excessive perspiration without sufficient liquid intake are other causes of dehydration, which may be particularly worrisome for athletes and people that work under hot, dry conditions. There are three main types of dehydration: hypotonic (primarily a loss of electrolytes, especially sodium), hypertonic (primarily a loss of water), and isotonic (equal loss of water and electrolytes). While isotonic dehydration is the most common, distinction between the three types of dehydration may be important for administering proper fluid replacement strategies.

Relying on thirst as a feedback mechanism to trigger demand for fluid intake may not be adequate to maintain an optimal hydration level, since a sensation of thirst sufficient to cause a subject to drink is often not triggered until after the subject is already dehydrated. Unfortunately, there are currently no practical, affordable, non-invasive devices for measuring a person's hydration level. Measurement devices that use blood or urine to measure hydration are impractical, invasive, and/or prohibitively expensive.

Many other physiological parameters and levels of various substances in the human or animal body are frequently tested or would be desirable to test for. Unfortunately, it is often necessary to sample blood, urine or other bodily substances, such as cerebrospinal fluid, to measure a desired parameter. Some physiological parameters involve even more invasive or costly measurement techniques.

Therefore, it would be highly beneficial to have a practical, affordable, non-invasive system and method for measuring a person's hydration status and quantifying the volume of fluid and electrolytes that need to be replenished following dehydration. It would also be very desirable to have practical, affordable, non-invasive systems and methods for testing other parameters in the body.

Point-of-care testing systems allow for measurement of biomarkers (e.g., metabolites, hormones, electrolytes) in biological samples outside of a laboratory, such as in a clinic or personal residence. By reducing labor and transport costs, point-of-care testing is an attractive alternative to laboratory testing, especially for frequent and/or routine tests.

Point-of-care testing systems typically consist of a handheld meter, which interfaces with a single-use test strip chemically responsive to an analyte (e.g., glucose). Typically, the handheld meter will perform all the steps necessary for sample analysis (signal generation, signal measurement, data processing) and display the result on an built-in screen. Some more advanced testing systems are capable of measuring multiple analytes using different test strips based on the same detection method (e.g., amperometric detection of glucose and beta-hydroxybutyrate) and/or communicating the test result wirelessly to a phone or tablet for data logging.

Currently available point-of-care testing systems are appropriate for individual users who monitor only one or two of their own biomarkers (e.g., a patient with diabetes who measures blood glucose each day). Such systems are less ideal, however, for large organizations, where many different biomarkers are assayed by multiple operators on tens or hundreds of different subjects (e.g., a hospital where hundreds of patients are tested each day for one or multiple biomarkers by multiple health professionals). For these organizations, test administrators must collect and curate results from many different devices, each measuring a different biomarker with a different detection method.

Therefore, it would be desirable to have an analysis system that is versatile enough to test for an extensive range of biomarkers using a single testing system. Ideally, such a test system would allow multiple users to conveniently administer and analyze test results while minimizing user error.

Sweat testing to determine the volume and composition of sweat is an increasingly popular method of generating personal rehydration strategies. This service is primarily used by athletes to determine their fluid replacement needs and gain a competitive edge. Typically, these services involve a one-off collection of activity-stimulated or chemically-stimulated sweat, through a patch affixed to the skin. The collected sweat is then assessed by a chemical analysis system to determine sweat sodium concentration (in millimoles or parts per million). These measurements are paired with exercise induced body mass change when following an exercise protocol to determine sweat rate and fluid loss (e.g., liters/hour). With this information, a personalized rehydration protocol is developed to assist in fluid replacement during and/or after exercise.

A key flaw of this methodology is the assumption that both sweat electrolyte content and sweating rate are consistent for an individual under all conditions. In fact, both sweat composition and rate can differ dramatically, based on many factors, including the activity being undertaken, the degree of exertion, the environmental conditions, and how acclimatized an individual is to these conditions. To ascertain this information for an individual, multiple measures are required over several conditions. With conventional approaches for estimation of sweat composition and sweat rate, this is prohibitively time consuming.

Therefore, it would be highly beneficial to have a practical, affordable, non-invasive system and method for measuring a person's hydration status and quantifying the volume of fluid and electrolytes that need to be replenished to prevent or treat dehydration. It would also be very desirable to have practical, affordable, non-invasive systems and methods for testing other parameters in the body. It would also be desirable to develop an accurate, rapid method of determining individual fluid volume and composition requirements on a case-by-case basis. Ideally, such a method would be relatively easy to employ and cost effective to make it accessible to many users. This application addresses at least some of these objectives.

### SUMMARY

Saliva may be an ideal bodily substance for use in measuring hydration and dehydration. Saliva is easily obtained with minimal invasiveness, but it is a complex fluid. Approximately 99% of saliva is water, and the remaining 1% comprises large organic molecules (such as proteins), small organic molecules (such as urea), and electrolytes (such as sodium and potassium). Whole saliva, considered as the total fluid content of the mouth, contains many other constituents, including serum components, blood cells, bacteria, bacterial products, epithelial cells, cell products, food debris and bronchial secretions. Thus, processing saliva to measure an individual's hydration level is challenging but likely highly beneficial if done effectively.

The assignee of the present application has filed previous patent applications describing systems, methods and devices for testing, measuring and analyzing saliva, to measure a subject's hydration level, as well as for measuring other substances and/or physiological parameters in a human or animal subject. These previous patent applications include U.S. patent application Ser. No. 16/197,530 (U.S. Pub No. 2019/0150836), titled “Saliva Testing System,” filed Nov. 21, 2018; and Ser. No. 16/598,000, titled “Ion Selective Sensor,” filed Oct. 10, 2019 (U.S. Pub No. 2019/0150836). The applications also include U.S. Provisional patent application Ser. No. 62/872,339, titled “Saliva Test Strip and Method,” filed Jul. 10, 2019; 62/961,438, titled “Assessment of Biomarker Concentration in a Fluid,” filed Jan. 15, 2020; and 62/967,694, titled “Biological Fluid Sample Assessment,” filed Jan. 30, 2020. All of the above-referenced patent applications are hereby incorporated by reference into the present application, and they are referred to collectively herein as “the Incorporated Applications.” The present application adds to the technologies in the Incorporated Applications by describing a biological fluid analysis system and method that addresses at least some of the objectives described above in the Background section.

The present application also adds to the technologies in the Incorporated Applications by describing a system and method for assessing hydration levels and electrolyte deficits and recommending hydration protocols before, during and after physical exertion. Specifically, this application describes a system and method of determining a personalized reference dataset, based on measurements of salivary osmolarity, body mass loss, sweat rate of exertion driven fluid volume and salt (electrolyte) loss under a variety of conditions. This reference dataset is used to establish an algorithm that can predict an individual's fluid and sodium replacement requirements, specific to environmental conditions, and may include biological measurements (e.g. salivary osmolarity), and degree of exertion, based on activity and/or standard rating of perceived exertion (RPE) scales. These requirements are then communicated to the individual to provide detailed guidance of when and what to ingest to offset fluid and sodium losses during exertion and to replenish and recover fluid and sodium losses following exertion or heat stress on a case-by-case basis.

In one aspect of the present disclosure, an analysis system includes a portable, handheld analyzer and one or more analyte specific test strips used to monitor multiple analytes from human or animal body fluids. The handheld analyzer can perform impedimetric, potentiometric and/or amperometric analysis and, using analyte-specific test strips, it can determine the concentration of multiple analytes. To improve ease of use, the handheld analyzer may automatically detect test strip type and batch and automatically apply temperature compensation to accommodate ambient conditions. In one embodiment, the handheld analyzer may be used independently in a stand-alone mode. In another embodiment, the handheld analyzer may be wirelessly connected to a phone, tablet or the like, to upload measurement data to a cloud database. The cloud database may be accessed using a phone or tablet to view and analyze data from multiple users and devices. To improve validity and accuracy, the system may integrate a method for measurement interpretation that is personalized to an individual user, a method for detection of abnormal readings, an error detection algorithm, and/or a method for compensating for temperature effects on measurement value.

In another aspect of the disclosure, a method of measuring an analyte in a bodily fluid sample and combining measurement data from multiple users may first involve initiating a wireless connection between a handheld analyzer and a smart computing device on which an analyte analysis application has been downloaded and inserting a test strip into the handheld analyzer. The method may also involve collecting a sample of a bodily fluid on the test strip, measuring, with the handheld analyzer, a concentration of at least one analyte in the sample, wirelessly communicating the measured concentration from the handheld analyzer to the smart computing device, and displaying the measured concentration on the smart computing device. Finally, the method may involve transmitting the measured concentration to a database, and organizing data including the measured concentration and at least one additional measured analyte concentration from at least one additional user on the database. The order of these method steps may be altered in various alternative embodiments.

In some embodiments, the method may further involve initiating a wireless connection between the smart computing device and the Internet, where the database is located on a cloud storage location, and where transmitting the measured concentration to the database involves wirelessly transmitting the measured concentration from the smart computing device to the cloud storage location via the Internet. In some embodiments, the data is organized based upon in groups of multiple users belonging to multiple organizations. In some embodiments, the method may also involve initiating the measuring step via the smart computing device, and initiating the measuring step may involve: logging into an operator account on the analyte analysis application; selecting a specific source from which the sample will be taken; and confirming the wireless connection between the handheld analyzer and the smart computing device.

The method may optionally further include automatically downloading and storing, on the handheld analyzer, a test strip type and batch data. For example, in some embodiments, automatically downloading and storing the test strip type and batch data may involve measuring a resistance-encoded test strip identification on the test strip and comparing the test strip identification with data in a memory of the handheld analyzer to determine the test strip type and batch data. Optionally, such a method may further involve communicating the test strip type and batch data to the smart computing device and alerting a user through an error message on the handheld analyzer and smart computing device if the test strip type is an unknown test strip type.

In some embodiments, the method may also involve preventing use of a used or faulty test strip by determining that the test strip has already been used or is faulty and prompting the user to discard the test strip on at least one of the handheld analyzer or the smart computing device. The method may also optionally involve providing instructions to a user regarding how to collect the sample, using the application and/or the handheld analyzer. Also optionally, the method may involve using the handheld analyzer for determining an ambient temperature, applying a detection technique based on the ambient temperature, and determining the concentration of the analyte(s) using batch specific calibration coefficients and the ambient temperature. The method may also involve determining, with the handheld analyzer, that a measurement is inaccurate by measuring a signal inconsistency and detecting an abnormally high signal or an abnormally low signal for the test strip. In some embodiments, the method may further involve using the smart computing device to analyze the measured concentration to assist in user interpretation. In some embodiments, the smart computing device refers a raw measured concentration to a previously established individual specific reference value.

In another aspect of the present disclosure, a method of measuring at least one analyte in a bodily fluid sample from a subject may involve: inserting a test strip into a handheld analyzer; collecting the bodily fluid sample on the test strip by bringing the test strip in contact with a body part of the subject where a bodily fluid is present; removing the test strip from contact with the body part after the handheld analyzer indicates that a sufficient amount of the bodily fluid sample has been collected; applying an electrical signal to the test strip; measuring, with the handheld analyzer, a response of a combination of the test strip and the bodily fluid sample to the applied electrical signal; analyzing the response with the handheld analyzer to determine that the bodily fluid sample is a valid sample; measuring a concentration of the at least one analyte in the bodily fluid sample; and displaying the measured concentration on the handheld analyzer and/or transferring the measured concentration to another device to display the measured concentration, generate further calculations and/or store the measured concentration. Again, the order of these steps may be altered without departing from the scope of the present invention.

In another aspect of the present disclosure, a handheld analyzer for determining a concentration of one or more analytes in a bodily fluid may include: a housing; a test strip port in the housing; a display screen on the housing; a temperature sensor in the housing; and multiple electronic components in the housing. The multiple electronic components may include: at least one of a direct digital synthesis (DDS) chip or a digital-to-analog converter (DAC) chip; an analog-to-digital converter (ADC) chip; a wireless communication chip; processing circuitry; and computer memory.

In some embodiments, the test strip port is configured to accept a test strip selected from the group consisting of analyte specific test strips and test strips capable of measuring multiple analytes. In some embodiments, the multiple electronic components are configured to automatically transfer test strip configuration settings to the computer memory when the handheld analyzer is connected to a database via a mobile application. In some embodiments, the handheld analyzer is configured to determine a test strip type and batch data using a resistance-encoded identification on a test strip and data stored in the computer memory. In some embodiments, the multiple electronic components are configured to automatically adjust a detection method, an excitation waveform and gain settings for multiple types of test strips. In some embodiments, the temperature sensor is configured to measure an ambient temperature, and wherein the processing circuitry is configured to process the measured ambient temperature using a temperature detection algorithm. In some embodiments, the multiple electronic components are configured to automatically detect and interpret application of a bodily fluid sample to a test strip, using a fluid detection algorithm, and at least one of initiate measurement or ensure sample consistency.

In some embodiments, the processor is configured to compare a measured concentration and a measured temperature to reference data specific to a test strip type and batch to determine analyte concentration. In some embodiments, the processor is configured to analyze raw measurement by an error detection algorithm to ensure sample consistency and measurement integrity. In some embodiments, the handheld analyzer is configured to display an error message on the display screen if a test strip is inserted while the handheld analyzer is being charged. In some embodiments, the processor is configured to count a number of measurements that have been conducted with the handheld analyzer and provide an alert to perform routine maintenance procedures on the device. In some embodiments, the handheld analyzer is configured to perform auto-calibration and self-testing to account for manufacturing variability.

In another aspect of the disclosure, a method for interpreting an analyte concentration for a subject may involve: taking at least one or measurement of an analyte under controlled conditions on at least one occasion from the subject; using an algorithm to determine a personalized reference range for the analyte specific to the subject under the controlled conditions; and using the personalized reference range to provide a specific measurement interpretation customized for the subject.

In some embodiments, the method may further involve using a protocol to establish a physiological state in which controlled measurements can be collected as desired by a user. In some embodiments, the method may further involve using a protocol to establish a physiological state in which measurements are specially outlined as part of the protocol. In some embodiments, the method may further involve outlining specific conditions in which controlled measurements may be conducted. In some embodiments, the method may further involve establishing multiple personalized reference ranges for a single analyte using multiple protocols. In some embodiments, the method may further involve using at least one personalized reference range from a single analyte to provide individual-specific interpretation of subsequent measurements. In some embodiments, the method may further involve using personalized reference ranges for multiple analytes to provide individual-specific interpretation of subsequent measurements.

In another aspect of the disclosure, a method for assessing measurement integrity may involve: applying at least one signal to a test strip by a handheld analyzer; monitoring the at least one signal for inconsistency, using the handheld analyzer; and classifying a measurement as normal or abnormal, based on whether there is inconsistency of the signal(s). In some embodiments, the signal includes multiple signals. signal comprises part of a measurement signal. In some embodiments, the signal is independent of a measurement signal. In some embodiments, measurement inconsistency is used to categorize a measurement as unreliable and the operator is directed to discard the measurement. In some embodiments, measurement inconsistency is used to categorize a measurement as unreliable and the measurement is not reported to the operator. Some embodiments may further involve using measurement inconsistency to categorize a measurement as unreliable and reporting an unreliable measurement to a user. Some embodiments further involve using measurement inconsistency to categorize a measurement as unreliable and automatically prompting a user to perform another measurement. Optionally, the method may further involve using measurement inconsistency to change at least one of a method of detection or a measurement interpretation algorithm applied by the handheld analyzer.

In another aspect of the present disclosure, a method of compensating for temperature when measuring an analyte may involve: monitoring temperature with a temperature sensor; using an algorithm housed in a handheld analyzer to determine ambient temperature; and using the determined ambient temperature to change at least one of signal generation parameters, signal detection parameters, or signal interpretation for measuring the analyte.

In some embodiments, temperature is monitored by the handheld analyzer with a built-in temperature sensor. In some embodiments, temperature is monitored by the handheld analyzer with an external temperature sensor. In some embodiments, a rate of change in temperature is used to determine the ambient temperature. In some embodiments, the method may further involve using at least one of the ambient temperature or a rate of change in temperature to determine a time need to equilibrate a sample to a target temperature before a detection method is applied.

In another aspect of the present disclosure, a method of generating a personalized reference dataset of fluid loss for a human subject may involve: following a protocol or set of protocols or conducting a series of exercise sessions; collecting first data related to a sweat salt content biomarker reflective of an amount of salt in sweat collected from the human subject; collecting second data related to a body mass change biomarker reflective of a change in a body mass of the human subject through fluid loss; and processing the first data and the second data with an algorithm to generate a reference dataset.

In various embodiments, the sweat salt content biomarker may include, but is not limited to, sweat osmolarity, sweat conductivity, and/or sweat electrolyte concentration. In various embodiments, the body mass change biomarker may include, but is not limited to, salivary osmolarity, salivary conductivity, salivary electrolyte concentration, urine osmolarity, urine specific gravity, urine color, and/or direct measurement of body mass. In some embodiments, the method may further involve calibrating a saliva biomarker for fluid loss using a set of paired measurements of changes in the saliva biomarker and fluid loss as determined by a change in weight of the human subject. In some embodiments, the saliva biomarker for fluid loss is measured before and/or after a protocol or exercise session, and the sweat biomarker is measured during and/or after the a protocol or exercise session.

In some embodiments, the protocol involves engaging in an activity for a set duration at a specific level of exertion, as determined by a self-perceived exertion scale or a heartrate-based metric of exertion, under at least one defined environmental condition. In some embodiments, the protocol involves engaging in a series of activity sessions, after which the human subject reports a level of exertion and at least one environmental condition. In some embodiments, the protocol is determined using parameters defined by the human subject. In some embodiments, the exertion level is automatically logged with a personal activity monitor. In some embodiments, a blood biomarker, a sweat biomarker and/or a saliva biomarker may be used to assess a degree of exertion. In some embodiments, the method further includes collecting at least one environmental condition via manual input from the human subject. In some embodiments, the method may further include collecting at least one environmental condition via automatic input from a weather monitoring service or device.

In another aspect of the present disclosure, a method of personalizing fluid replacement guidelines for a human subject after the human subject has engaged in an activity, in which a set of reference values of fluid loss volume and salt content have been previously established for the human subject, may involve: using a body mass change biomarker of fluid loss to establish a change in body mass of the human subject through fluid loss related to a degree of exertion and at least one environmental condition; using an algorithm to predict an amount and a chemical composition of fluid lost from the human subject; and providing advice to the human subject on a volume and a composition of fluids required for replacement and recovery and a time period over which the fluids should be ingested.

In various embodiments, the body mass change biomarker may include, but is not limited to, salivary osmolarity, salivary conductivity, salivary electrolyte concentration, urine osmolarity, urine specific gravity, urine color, and/or direct measurement of body mass. In some embodiments, the degree of exertion is self-reported by the human subject. In some embodiments, the degree of exertion is established using a personal activity monitor or a heart rate monitor. In some embodiments, the method may further involve using a biomarker such as but not limited to a blood biomarker, a sweat biomarker and/or a saliva biomarker, to determine the degree of exertion.

In some embodiments, the environmental condition is self-reported by the human subject. In some embodiments, the environmental condition is determined using a weather monitoring service or device. In some embodiments, the advice is provided via a computer application for at least one of a smart phone or a tablet computing device. In some embodiments, providing the advice involves providing prompts to the human subject via the computer application regarding when to consume the fluids and at least one type of the fluids to drink.

In another aspect of the present disclosure, a method of personalizing fluid replacement guidelines for a human subject engaging in an activity, in which a set of reference values of fluid loss volume and salt content have been previously established for the human subject, may involve: establishing a degree of exertion for the human subject engaging in the activity; establishing at least one environmental condition; predicting, using an algorithm with the established degree of exertion and at least one environmental condition, an amount and a chemical composition of fluid lost by the human subject during the activity; and providing advice to the human subject regarding a volume and a composition of fluids required for recovery and a time period over which the fluids should be ingested.

In another aspect of the present disclosure, a method of personalizing fluid replacement guidelines for a human subject before engaging in an activity, in which a set of reference values of fluid loss volume and/or salt content have been previously established for the human subject, may involve: establishing at least one environmental condition; establishing a degree of exertion as predicted by the human subject; using an algorithm with the at least one environmental condition and the degree of exertion to predict an amount and a chemical composition of fluid lost by the human subject during the activity; and providing advice to the user regarding a volume and a composition of fluids for maintaining hydration during exercise and for recovery after exercise for the human subject. In some embodiments, the method may further involve using a hydration biomarker to establish a hydration state of the human subject before engaging in the activity and using the hydration biomarker to customize the advice.

In another aspect of the present disclosure, a method of personalizing fluid replacement guidelines for a human subject after engaging in an activity, in which a set of reference values for fluid loss volume and/or salt content have been previously established for the human subject, may involve: measuring salivary osmolarity after the exercise event; and providing advice to the user regarding a volume and a composition of fluids for recovery after exercise for the human subject.

Optionally, the method may further involve using a hydration biomarker, such as salivary osmolarity, to establish a hydration state of the human subject after engaging in the activity, and using the hydration biomarker to customize the rehydration advice. In other embodiments, the method may involve using a hydration biomarker to establish a hydration state of the human subject after engaging in the activity and using longitudinal (temporal) hydration biomarker markers to customize and adapt the advice based on how quickly the human subject's hydration is returning to a desirable level. In other embodiments, the method may involve using a hydration biomarker to establish a hydration state of the human subject after engaging in the activity and using longitudinal (temporal) hydration biomarker markers to customize and adapt the advice based on how quickly the human subject's hydration is returning to a desirable level. The time between hydration biomarker measurements is determined by the difference between the desired and current hydration status.

In another aspect of the present disclosure, a method for determining a sodium content of a sweat sample from a human subject may involve: collecting the sweat sample from the human subject; performing an electrochemical test on the sweat sample using a portable, handheld testing system to take a measurement of at least one of conductivity, impedance or osmolarity of the sweat sample; and converting the measurement into the sodium content using a calibration curve. In some embodiments, collecting the sweat sample involves collecting a small volume of sweat directly from the human subject's skin with a single-use test strip of the handheld, portable testing system. In some embodiments, collecting the sweat sample involves: collecting sweat from the human subject's skin via an adhesive patch directly applied to the skin; extracting the sweat from the patch; and collecting the sweat sample from the extracted sweat with a single-use test strip of the handheld, portable testing system.

In some embodiments, the sweat sample is collected using an adhesive patch that includes at least one of electrodes and microfluidics, and the adhesive patch provides on-skin analysis of the sodium content through a physical connection with the handheld, portable testing system. In some embodiments, the sweat sample is collected using an adhesive patch that includes electrodes, microfluidics and/or electronic components, and the adhesive patch performs the electrochemical test and wirelessly communicates raw data from the electrochemical test to the handheld, portable testing system. In some embodiments, the portable testing system is further configured to perform analysis of at least one additional biomarker of sweat, saliva or blood, to establish a body mass change and/or a physical exertion of the human subject.

These and other aspects and embodiments are described in greater detail below, in relation to the attached drawing figures.

### BRIEF DESCRIPTION OF THE DRAWINGS

FIG. 1 is a diagram of a biological fluid analysis system, according to one embodiment;

FIG. 2 is a diagram of a handheld analyzer, according to one embodiment;

FIG. 3 is a flow chart of a method for using a biological fluid handheld analyzer independently as a point-of-care device, according to one embodiment;

FIG. 4 is a flow chart of a method for using a biological fluid handheld system, including pairing with a phone or tablet, according to one embodiment;

FIG. 5 is a diagram of a system for creating a reference dataset for hydration and sweat loss for a human subject, according to one embodiment;

FIG. 6 is flow chart illustrating a method for assessing sweat loss and recommending a rehydration protocol, according to one embodiment;

FIG. 7 is flow chart illustrating a method for assessing sweat loss and recommending a rehydration protocol, according to an alternative embodiment;

FIG. 8 is flow chart illustrating a method for assessing sweat loss and recommending a rehydration protocol, according to another alternative embodiment;

FIG. 9 is a flow chart illustrating a protocol for rehydrating and monitoring hydration after exercise;

FIG. 10 is a flow chart illustrating a fluid replacement algorithm based on measurement of salivary osmolarity;

FIG. 11 is a flow chart illustrating a sodium replacement algorithm based on measurement of sweat sodium concentration;

FIG. 12 is a flow diagram illustrating a method for assessing sweat loss, recommending a rehydration protocol, and feeding data back to a reference dataset, according to another embodiment;

FIG. 13 is a system drawing, illustrating a sweat collection and analysis system, according to one embodiment;

FIGS. 14A-14D illustrate a method for collecting and analyzing sweat, according to one embodiment;

FIG. 15 is a chart, illustrating correlation between osmolarity and sodium concentration of sweat samples, according to one embodiment; and

FIG. 16 is a flow diagram illustrating a method for assessing sweat loss, recommending a rehydration protocol, and feeding data back to a reference dataset, according to another embodiment.

### DETAILED DESCRIPTION

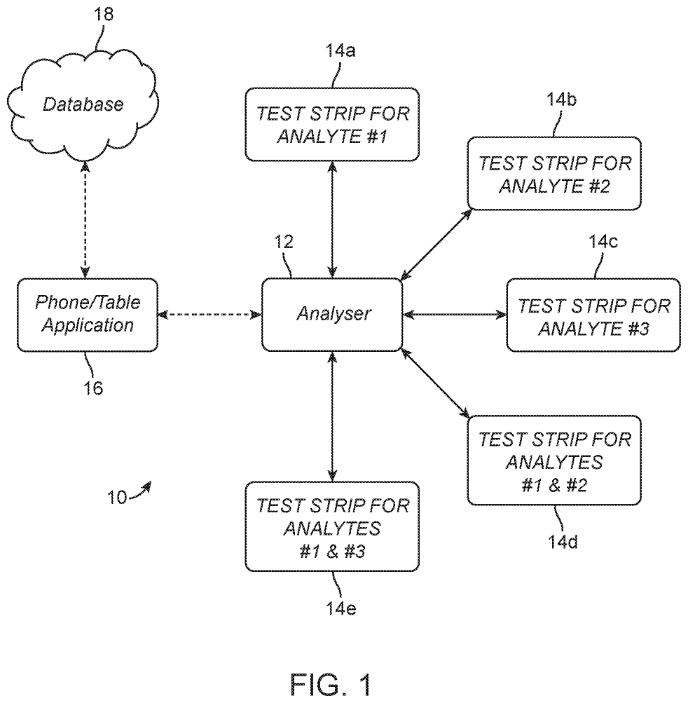

The present application describes various embodiments and features of a biological fluid analysis system and method. Referring to FIG. 1, one embodiment of a biological fluid analysis system 10 includes a handheld analyzer 12, multiple test strips 14a-14e, and an application 16 for use on a phone, tablet or other smart computing device. The system 10 may further include a database 18 (or multiple databases) for storing data, which the application 16 may access. The database 18 may be located in the cloud or any other suitable location.

Test Strips

In various embodiments, the biological fluid analysis system 10 may include any suitable number and combination of types of test strips 14a-14e. In some embodiments, for example a panel of test strips 14a-14e may be provided, with each strip 14a-14e being chemically sensitive to a specific analyte (e.g., electrolytes, metabolites, hormones) or a panel of analytes (e.g., multiple electrolytes). The test strips 14a-14e are single use, disposable and configured to test for a specific biological sample type (e.g., blood, saliva, sweat, urine) or non-biological sample type (e.g., pool water, wastewater). Test strips 14a-14e may be visually distinguishable in some embodiments and/or may contain a resistor-encoded identification code. Test strips 14a-14e may use one of multiple of a range of detection methods.

Test strips 14a-14e may significantly differ in size, shape and design, but share common design elements allowing for compatibility with a single analyzer 12. In one embodiment, a test strip 14a-14e includes a sampling port and four untreated carbon electrodes, three of which are used for impedimetric measurement and the fourth of which is the resistance-encoded identification code describing the test strip type and batch. In another embodiment, the test strip 14a-14e includes a sampling port and three carbon electrodes, two of which are configured to allow for potentiometric measurement of an analyte, and the third of which is the resistance-encoded identification code describing the test strip type and batch. Common to both of these embodiments of test strips 14a-14e is the electrode structure for interfacing with the handheld analyzer 12 and test strip identification. All other features are configured for a given analyte (or set of analytes) and sample type.

In the embodiment illustrated in FIG. 1, four types of test strips 14a-14e are shown as part of the biological fluid analysis system 10. (Multiple strips of each type may be provided, and alternatively any other suitable number of test strip types may be provided.) In this embodiment, the analysis system includes a test strip for analyte #1 14a, a strip for analyte #2 14b, a strip for analyte #3 14c, a strip for analytes #1 and #2 14d, and a strip for analytes #1 and #3 14e. Of course, analytes #1, #2 and #3 may be any suitable analytes, according to various embodiments.

Analyzer

In some embodiments, the analyzer 12 of the biological fluid analysis system 10 is a handheld, point-of-care analyzer 12 capable of signal generation, measurement and processing. The handheld analyzer 12 may automatically determine the type of test strip 14a-14e inserted into it, for example by reading a resistor-encoded identification code on the test strip 14a-14e. The handheld analyzer 12 may use the identification information to configure the detection method, excitation waveform and gain settings, and to apply a batch specific calibration curve when processing the raw measurement data.

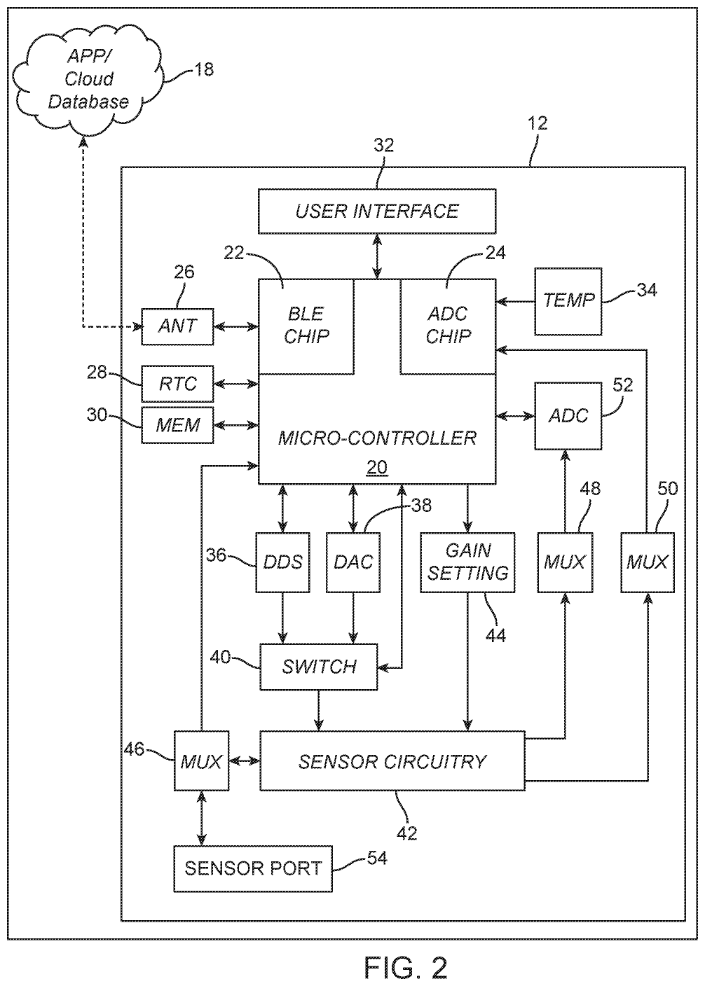

Referring now to FIG. 2, one embodiment of the handheld analyzer 12 is illustrated in detail. In this embodiment, the handheld analyzer 12 includes a micro-controller 20 with an onboard Bluetooth low energy chip 22 for wireless communication and an analog-to-digital converter (ADC) chip 24 for measurement of analog signals. The analyzer further includes: an antenna 26 for wireless communication; a real time clock 28; memory 30 for storage of test-strip identification and batch calibration data; a user interface 32 including a screen, buttons and LED; a temperature sensor 34 for monitoring of ambient temperature; a direct digital synthesis chip 36 for digital signal generation; a digital-to-analog converter chip 38 for analog signal generation; a low-noise analog switch 40 for regulating the transfer of signal generation to sensor circuitry 42; and gain setting circuitry 44.

The handheld analyzer may also include three multiplexers (“MUX”) 46, 48, 50. The first multiplexer 46 is configured to regulate the connection between the sensor circuitry 42 and a sensor port 54. The second multiplexer 48 is configured to regulate the connection between the sensor circuitry 42 and a high-resolution ADC 52. The third multiplexer 50 is configured to regulate the connection between the sensor circuitry 42 and the micro-controller 20.

The features of this embodiment of the handheld analyzer 12 allow a diverse range of analytical techniques to be employed with a single analyzer 12. Specifically, the multiplexing of signal generation, gain settings and a second high-resolution ADC allow for impedimetric, amperometric and potentiometric analysis.

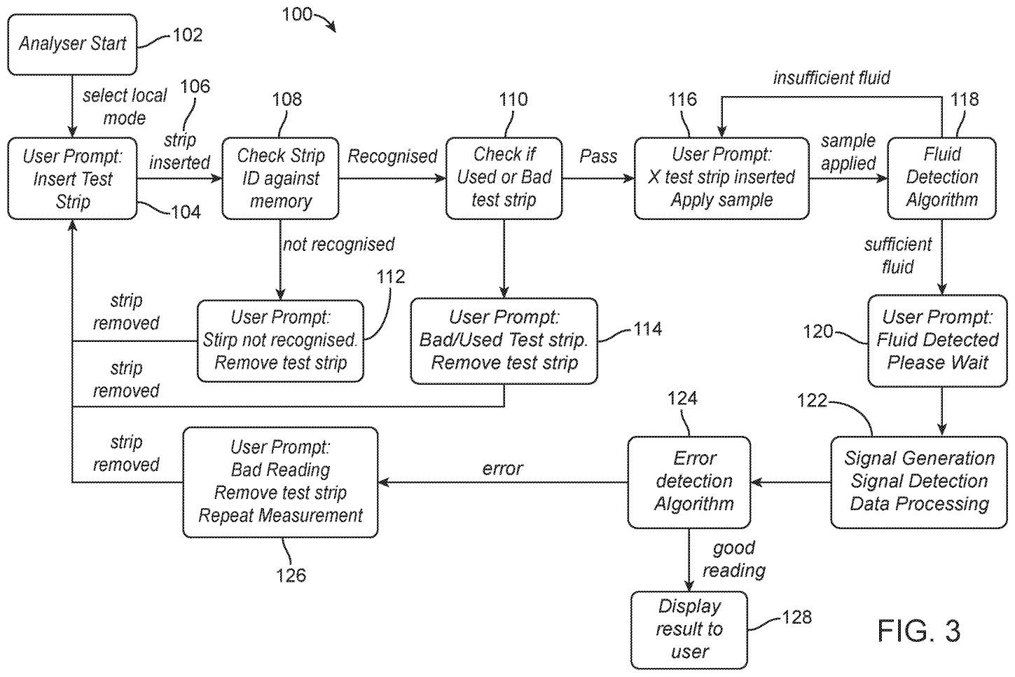

FIG. 3 illustrates one exemplary method 100 for using the handheld analyzer 12 independently, as a point-of-care analysis device. In this embodiment, the method begins by starting (e.g., powering on) the analyzer 12 in step 102. In step 104, the user is prompted to insert a test strip. The user inserts a test strip into the analyzer 12 in step 106, and the handheld analyzer 12 automatically detects, in steps 108 and 110, the test strip type and batch to ensure that the test strip is not used or faulty. For example, in one embodiment, the impedance of a test strip is used to determine if it is used or faulty. In another embodiment, a one-way fuse is used to ensure a test strip is not reused.

An error detection algorithm may used to identify suspect readings. In one embodiment, a periodic stimulus signal is applied and consistency between measurements used to assess accuracy of results. In another embodiment, this periodic signal is applied at multiple distinct frequencies and these are investigated. A reference range of values may also be used to determine whether a result is of an appropriate range for a given analyte or sample type.

If step 108 or step 110 indicate a “bad” strip has been inserted, the analyzer provides the user with an error code or other prompt (steps 112 and 114), so that the user knows to remove the test strip from the analyzer 12 and start the method again. If the test strip is accepted by the analyzer 12, the user receives a prompt in step 116 to apply a fluid sample to the test strip. Instructions for sample collection and error messages may be relayed to the user on a built-in display screen. In step 118, the analyzer 12 applies a fluid detection algorithm to the fluid sample to determine if the sample is adequate. If the sample is insufficient, the user receives another prompt (repeating step 116) to add more fluid to the test strip. If the sample is sufficient, the analyzer may provide another prompt to the indicate that fluid was detected and to please wait for results (step 120). Data processing is performed on the handheld analyzer 12 in step 122, and an error detection algorithm is applied in step 124. If the analyzer 12 detects an error, the user is prompted accordingly in step 126, and the test strip is removed and replaced with a new strip to start the method again. If the error detection algorithm confirms a good reading of the fluid sample, the results of the measurement are displayed to the user on the analyzer and/or a smart device coupled with the analyzer in step 128. In some embodiments, the handheld analyzer 12 may include integrated cellular or wireless capability. In such embodiments, the handheld analyzer 12 can upload measurement results for storage in a cloud server or other database.

The handheld analyzer 12 may record the number of measurements performed on its internal memory. Using this information, the user is prompted to perform routine maintenance at specific milestones. In one embodiment the user is prompted to replace the test-strip port after a certain number of measurements have been performed. In some embodiments, the handheld analyzer also contains an internal reference load, which may be used to perform start-up calibration and account for manufacturing variability.

Phone/Tablet Application

Referring back to FIG. 1, the application 16 provided with the biological fluid analysis system 10 may be configured for use on a smart phone, tablet and/or other computing device. The application 16 facilitates step-by-step operator instructions, subject selection and measurement interpretation. Measurements made by the handheld analyzer 12 may be automatically uploaded to a cloud database 18 via the application 16.

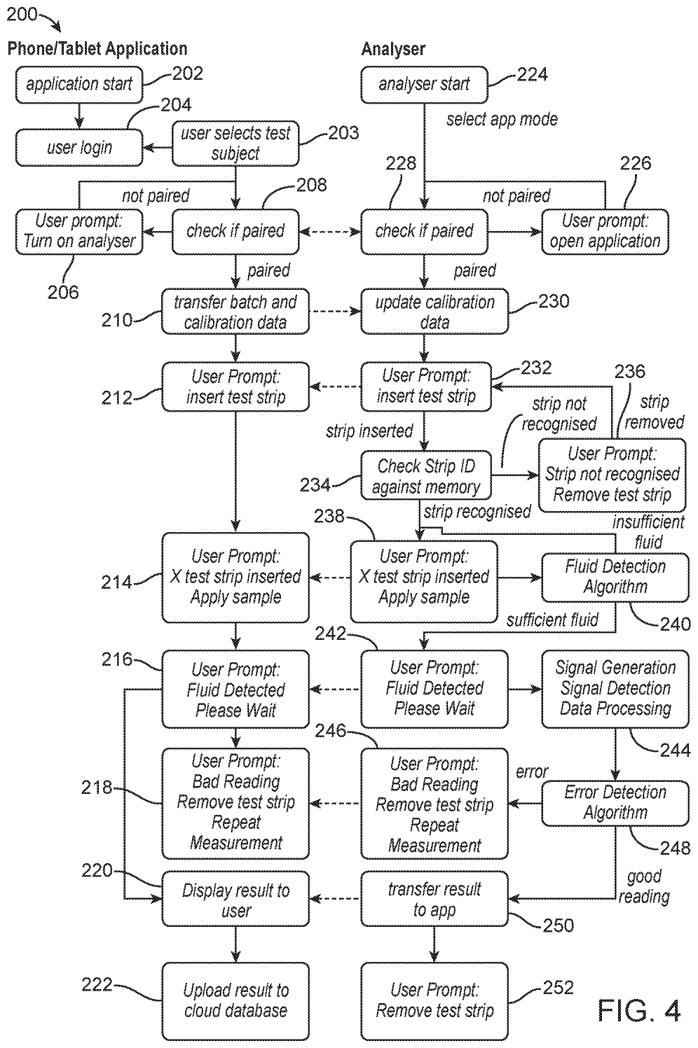

Referring to FIG. 4, a method 200 of operation of the phone/tablet application with the handheld analyzer 12 is illustrated. In this method, application is started 202 and the user logs in 204. The user selects a test subject 203. The app prompts the user to turn on the analyzer 206. The user starts the analyzer 224, which prompts the user to turn on the app 226. Obviously, the order of these steps may be reversed, and the prompts are optional. The app checks to see if it is paired with the analyzer 208, and the analyzer checks to see if it is paired with eh app 228, using a data logging application. Upon pairing, any new type/batch data may be transferred to the handheld analyzer memory (steps 210 and 230). The user then selects a profile to initiate a measurement.

Next the user is prompted to insert a test strip into the handheld analyzer (steps 212 and 232), the user inserts the test strip, and the analyzer walks the user through steps similar to or the same as those described in relation to FIG. 3 (steps 234, 236, 238, 240, 242, 244, 246, 248). Instructions may be displayed on the handheld analyzer and/or phone/tablet, instructing the user as to how to collect a sample. (See, for example, steps 216, 218, 220 regarding display on app). Sample analysis and data processing are performed on the handheld analyzer. Ambient temperature may be used to adjust the measurement technique or assist in processing of data. Results are communicated to the application (step 250), displayed on the phone/tablet (step 220) and logged to the user profile. Interpretive information may be displayed on the phone application screen. Results may be automatically uploaded to a cloud server (step 222), if an Internet connection is available and/or stored locally for upload when a connection is available. Finally, the user may be prompted to remove the used test strip 252.

In some embodiments, a user specific reference panel may be previously established through a protocol or set of protocols. This information may be used to provide user specific interpretive information.

Database

Referring again to FIG. 1, the biological fluid analysis system 10 may also include one or more databases 18 for storing any suitable data relevant for the system 10 and method. In some embodiments, the system 10 may include a database 18 located in the cloud. The database 18 may be decentralized and may store information for operation of the system 10, including test strip characteristics (type, batch, calibration coefficient), operator profiles, subject profiles and test results. The cloud database 18 can be accessed by operators through a web browser to review, analyze and export test results. Users with necessary permissions may view test results collected by multiple users and/or multiple analyzers for multiple tests.

The present application describes various embodiments and features of a hydration assessment system and method, for determining a human subject's level of hydration and recommending a hydration protocol. Although the following disclosure focuses on the analysis of sweat and/or saliva, the embodiments described below, or variations of those embodiments, may be used for analysis of any other bodily fluid, such as blood, urine or the like.

The Reference Dataset

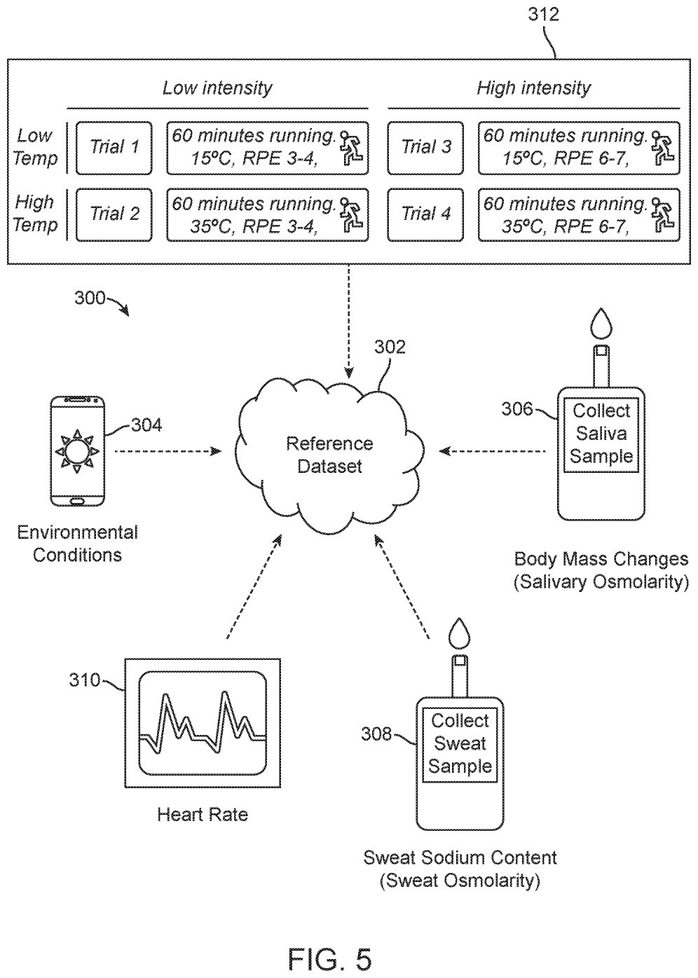

FIG. 5 shows a hydration measurement system 300 for creating a reference dataset 302 of information related to sweat loss and hydration of a human subject. The reference dataset 302 includes a panel of measured parameters collected across multiple measurement sessions 312, using one or more measurement devices. The parameters may include, but are not limited to, environmental conditions 304 (e.g., temperature, humidity, wind-speed, elevation), body mass changes and/or salivary osmolarity changes 306, sweat electrolyte content 208, and/or one or more physical exertion measurements 310 (e.g., heart rate, respiratory rate, etc.). Each of the various parameters 304, 306, 308, 310 may be measured using its own, separate measurement device or, in some cases, using one measurement device for measuring multiple parameters. For example, as illustrated diagrammatically in FIG. 5, a smart phone may be used for collecting environmental condition data 304, a saliva measurement device may be used for measuring body mass changes and/or salivary osmolarity 306, a sweat measurement device may be used for measuring sweat osmolarity/sweat sodium content 308, and a heart rate monitor may be used for measuring physical exertion 310. In an alternative embodiment, for example, saliva measurements 306 and sweat measurements 308 may be collected using the same handheld measurement device and the same or different test strips. These types of devices are described in more detail in the Incorporated References.

Environmental condition data 304 may be collected, for example, by manually logging parameter data from external equipment, automated data logging from a purpose-built testing system, or automated logging of these parameters from a weather monitoring service based on time and location data. The degree of physical exertion 310 may be determined, for example, through self-reporting on a standard rating of perceived exertion (RPE) rating scale and/or may be inferred from one or more measured biomarkers of physical exertion (e.g., activity, heart rate, VO2 max, lactic acid concentration in blood, sweat or saliva). These measurements may be manually reported and/or automatically logged with a personal monitoring device, such as a fitness watch or heart rate chest-strap. The percentage body mass loss 306 may be established, for example, through direct measurement of body mass before and after a period of exertion, accounting for any fluid ingested or lost through urination or inferred from a biomarker of change in body mass (e.g., increased salivary osmolarity or salinity, urine osmolarity or urine specific gravity). The sweat sodium content 308 may be established through direct measurement of the sweat sodium content 308 with a chemical analysis system or estimated from the conductivity or osmolarity of a sweat sample.

Thus, the hydration assessment system 300 may include multiple data capturing devices (or programs or applications on devices), to gather, for example, the environmental conditions 304, the body mass loss 306, the sweat sodium content 308 and/or the heart rate or other measure of exertion 310. Each measurement device is used over multiple measurement sessions 312 to collect the various types of data 304, 306, 308, 310, and provide the data to the reference dataset 302, which may be located in a database stored on a computer or in the cloud. The reference dataset 302 may be generated, for example, by following a set of predefined exercise protocols that specify duration and intensity of exertion prior to collection of measurements or by taking measurements over time during regular activity. In the illustrated example, data is collected over four trial measurement sessions 312 (trials 1-4). The human subject runs for 60 minutes during each session, at different outdoor temperatures and with different RPEs. Collected data from all four measurement sessions 312 feeds into the reference dataset 302. The reference dataset 302 may then be used to establish an algorithm, which may be used to estimate sweat rate and/or sweat sodium composition of the human subject under various conditions.

In the embodiment of FIG. 5, a set of four exercise trial measurement sessions 312 is defined, outlining environmental conditions (cool and hot) and intensity (RPE 3-4 (Light-Moderate Activity) and RPE 7-8 (Vigorous Activity). Environmental conditions 304 are manually logged by the user into a phone application. Salivary osmolarity 306 is measured before and after exercise with a handheld testing device (such as the device described in the Incorporated Applications), which wirelessly communicates the results to the phone application. The change in salivary osmolarity 306 may be used to estimate a change in body mass during each trial. Sweat may be collected with an adhesive patch (or other collection device in alternative embodiments) during each trial. Sweat osmolarity 308 may be measured with a handheld testing device (such as the device described in the Incorporated Applications), which wirelessly communicates the results to the phone application. Sweat osmolarity 308 or conductivity may be used to estimate sweat sodium content. Heart rate may be monitored with a chest strap monitor and wirelessly communicated to the phone application. Average heart rate may be used to determine the intensity of exertion 310.

Fluid Replacement Guidelines

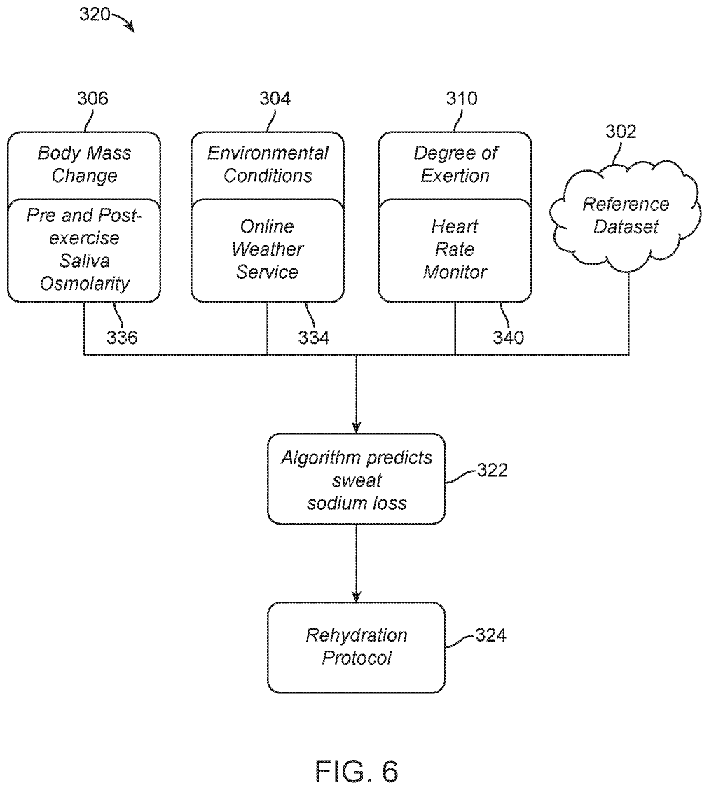

Referring now to FIG. 6, a method 320 is outlined for using the reference dataset 302 to generate a rehydration protocol 324 including fluid replacement guidelines for the human subject. These guidelines may assist in offsetting fluid and sodium losses during heat stress or physical exertion and to replenish and recover fluid and sodium back to rest levels following exertion or heat stress. Once the reference dataset 302 is established, the method 320 may involve using an algorithm 322 to predict sweat sodium loss and then using the predicted sweat sodium loss to generate a rehydration protocol 324. The instructions in the rehydration protocol 324 may define the volume of fluid required to replace losses, the sodium composition of fluid required to replace losses, and/or the time over which these fluids should be ingested. The algorithm may be implemented 322, and the rehydration protocol 324 may be generated, via a processor stored in a computer, an application stored on a smart phone or smart tablet, a processor in the cloud, or the like. An application may provide alerts to assist in following this protocol.

In the embodiment illustrated in FIG. 6, environmental conditions 304 are monitored with a portable weather monitoring system 334 and wirelessly communicated to a phone application. Exertion level 310 during an exercise session is monitored with a chest-strap heart rate monitor 340 and wirelessly communicated to a phone application. Salivary osmolarity 306 is measured before and after exercise with a handheld testing device 336, which wirelessly communicates the results to the phone application. The change in salivary osmolarity 306 is used to estimate change in body mass during exercise. The reference dataset 302 is used to predict the sweat composition 322 based on the recorded information. The phone application generates a rehydration protocol 324 outlining what and how much to drink to assist in rehydration and recovery after exercise.

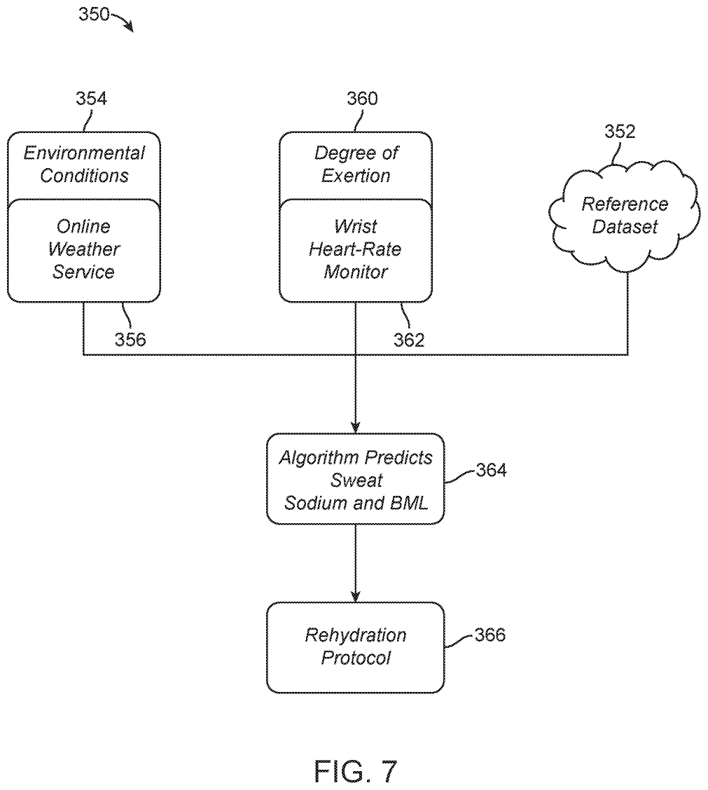

FIG. 7 illustrates another embodiment of a method 350 for assessing hydration and providing a rehydration protocol 366. In this embodiment, environmental conditions 354 are determined using an online weather monitoring service 356 and the user's location. Exertion level 360 during an exercise session is monitored with a heart rate monitor 362, for example a wrist-based monitor integrated into a smart watch, and wirelessly communicated to a phone application. The reference dataset 352 is used to predict changes in body mass and sweat composition 364, based on the recorded and measured information. The phone application generates a rehydration protocol 366 outlining what to drink to assist in recovery after exercise and periodically alerts the user through mobile notifications to reinforce drinking behavior. Again, the sweat composition predicting step 364 and the generating a rehydration protocol step 366 may be performed by any suitable processor, such as an application for a smart device, a processor in a computing device, a cloud processor or the like.

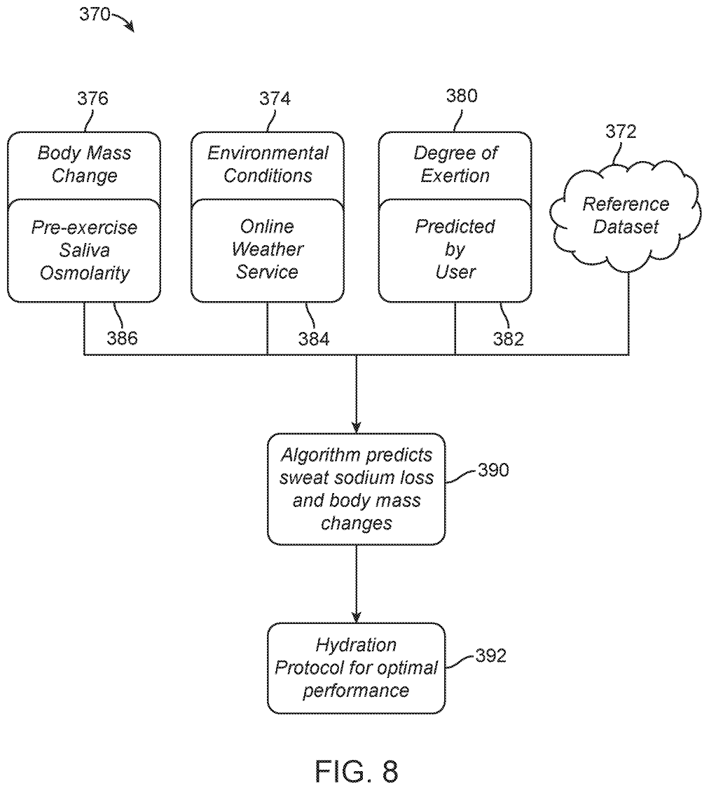

FIG. 8 illustrates another embodiment of a method 370 for assessing hydration and providing a rehydration protocol 392. In this embodiment, environmental conditions 374 are determined using an online weather monitoring service 384 and the user's location. Exertion levels 380 are predicted by the user specifying 382 anticipated intensity of exercise (e.g., race day/high intensity). Salivary osmolarity 376 is measured before exercise with a handheld testing device 386, which wirelessly communicates the results to the phone application. Salivary osmolarity 376 is used to estimate starting hydration status. The reference dataset 372 is used to predict changes in body mass and sweat composition 390, based on the recorded information. The phone application generates a rehydration protocol 392 outlining what to drink to assist in maintaining hydration during exercise and assist in rehydration and recovery after exercise. The user is periodically alerted during exercise to drink to assist in maintaining a hydrated state and to offset fluid losses.

In another embodiment, salivary osmolarity is measured post-exercise. The reference dataset together with final salivary osmolarity, or changes of salivary osmolarity before and after the event, or difference between an individuals optimal hydration zone, is used to predict changes in body mass. Sodium and electrolyte loss are estimated from sweat composition based on the recorded information. The phone application generates a protocol outlining what to drink, how much to drink and when to drink assist in hydration recovery after exercise.

In another embodiment, the user may be periodically alerted during post-exercise recovery to measure their salivary osmolarity. These other salivary measurements are used to estimate the effectiveness of the initial hydration protocol and to permit the hydration protocol to be adapted in order to increase its effectiveness and to return the individual to a desirable hydration status.

In another embodiment, the user may be periodically alerted during post-exercise recovery to measure their salivary osmolarity. The time period between prompts is based upon how far the individual salivary osmolarity and hydration status is from their desirable hydration status.

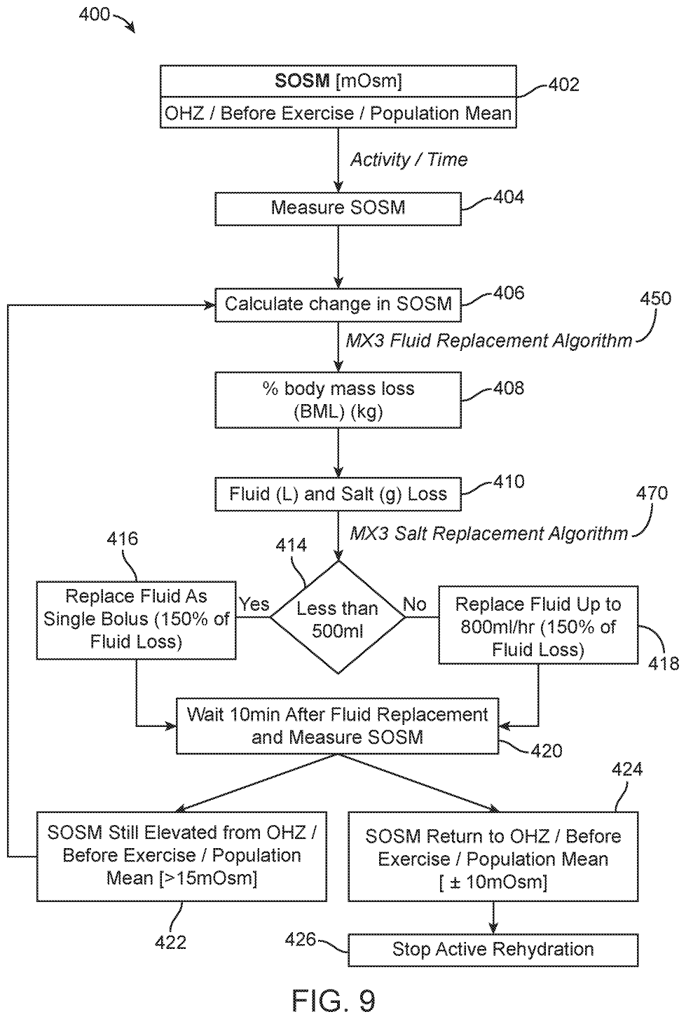

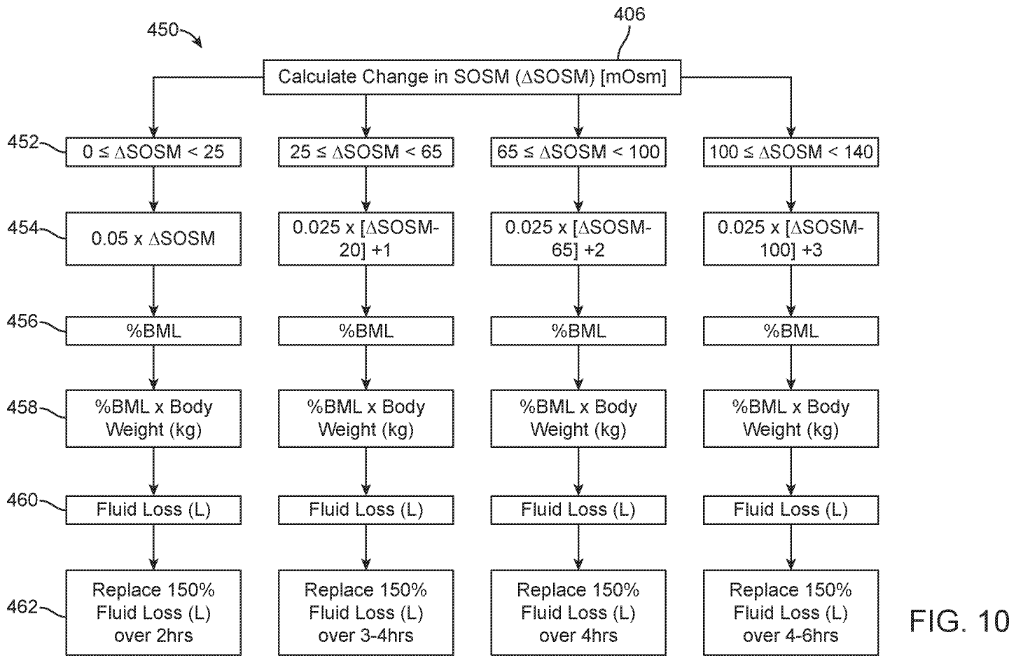

FIG. 9 illustrates one embodiment a rehydration protocol 400 (or “recovery protocol”). In this embodiment salivary osmolarity is measured before exercise 402 and after exercise 404, the change in osmolarity is calculated 406, and that change is used to estimate body mass loss 408 using a fluid replacement algorithm 450, such as the algorithm 450 illustrated in FIG. 10. Sweat sodium is either directly measured 410 or estimated from the reference panel using environment and/or exertional parameters, and a protocol for salt replacement generated 412 using a sweat replacement algorithm 470, such as the algorithm 470 illustrated in FIG. 11. Salivary osmolarity is used to monitor rehydration until the individual has appropriately achieved their desirable hydration status.

In the embodiment illustrated in FIG. 9, the rehydration protocol first involves the step of determining whether the fluid loss was less than 500 milliliters 414. If less than 500 mL, then the protocol instructs the user to replace fluids as a single bolus in the amount of 150% of fluid lost 416. If the fluid loss was greater than or equal to 500 mL, the user is instructed to replace fluids up to 800 mL/hour, up to 150% of fluid lost 418. The protocol next involves waiting approximately 10 minutes after fluid replacement and measuring salt osmolarity 420. If the osmolarity is still elevated compared to the pre-exercise measurement 422, then the method returns to step 406. If the osmolarity has returned to the pre-exercise level 424, then the rehydration protocol stops 426. Of course, this is just one example.

FIG. 10 illustrates one embodiment of a fluid replacement algorithm 450 that may be used as part of the rehydration protocol generation methods described herein. In this embodiment, the fluid replacement algorithm 450 calculates the change in saliva and/or sweat osmolarity 406, or alternatively the algorithm 450 may receive the change on osmolarity, after it has been calculated. The algorithm then categorizes the change in osmolarity into one of four categories 452, based on the size of the change. Then, the change is multiplied by a multiplier 454 and a percent of body mass loss is calculated 456. The body mass loss is multiplied by the user's body weight 458 to calculate fluid loss 460, and the fluid loss 460 is used to generate a fluid replacement protocol 462.

FIG. 11 illustrates one embodiment of a salt replacement algorithm 470 that may be used as part of the rehydration protocol generation methods described herein. In this embodiment, the salt replacement algorithm 470 may start with environment data and/or exertion data 472 and sweat data from a reference panel 476. Alternatively, it may start with measured sweat 474. The algorithm 470 calculates salt loss 478 based on fluid loss and sweat data. The algorithm 470 then divides the salt loss across a fluid replacement time 480 and determines whether salt loss has occurred at greater than 1200 mg/hour or less than or equal to 1200 mg/hour 482. The algorithm 470 then generates a salt replacement protocol 484, based on the amount of salt loss.

Testing Method

Currently, chemical analysis of sweat is performed using laboratory tools. These are bulky and expensive and require large samples for analysis. As the reference dataset described above requires multiple sweat measurements to be performed across multiple training sessions, laboratory-style analysis of samples may be impractical. To facilitate the methods described herein for hydration assessment and hydration recommendations, this application also describes a method of rapid assessment of sweat sodium content through measurement of sweat conductivity, impedance or osmolarity, using a handheld portable testing system.

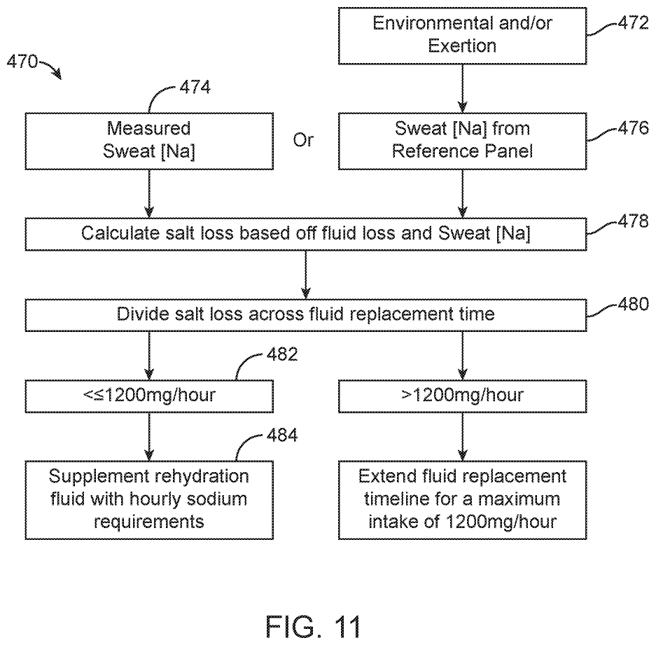

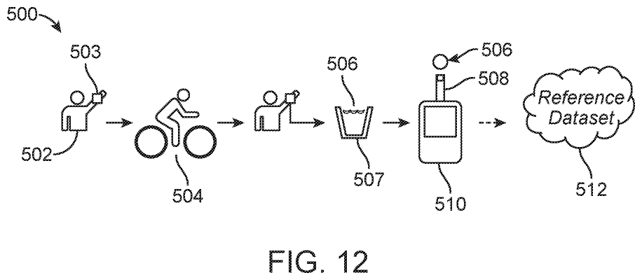

Referring now to the diagrammatic flow chart of FIG. 12, one embodiment of a method 500 for measuring a sweat sample is illustrated. In this embodiment, the method 500 involves a user 502 affixing an adhesive, absorbent patch 503 to the skin. The user then exercises 504 (or otherwise exerts himself/herself) and collects sweat with the patch 503. After exercise, sweat 506 is extracted from the patch 503, for example by squeezing the sweat 506 out of the patch 506 and into a collection receptacle 507. A small sample of the sweat 506 is then collected from the collection receptacle 507, using a single-use disposable test strip 508. Next, the test strip 508 is inserted into a portable, handheld testing system 510 (such as the system described in the Incorporated Applications). The testing system 510 then calculates sweat sodium concentration from impedance (or osmolarity) of the sweat sample 506. In alternative embodiments, the test strip 508 may be inserted into the handheld testing system 510 before applying the sweat 506 to the test strip 508. Once the testing system 510 has been used to generate any desired data pertaining to the sweat 506, some or all of the data may be sent (wirelessly, for example) to the reference dataset 512 for the user.

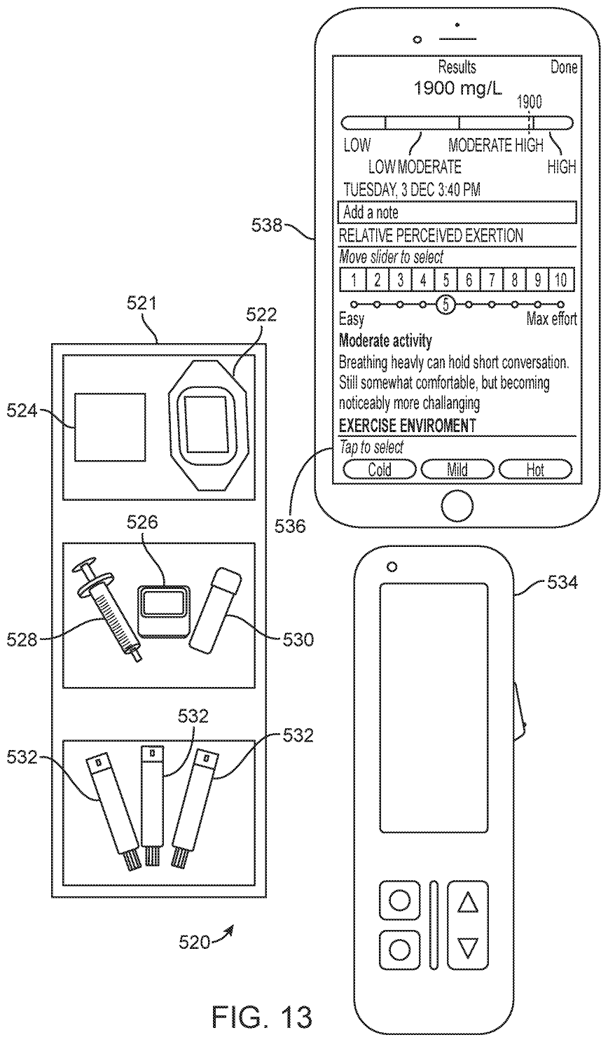

FIG. 13 is an illustration of one embodiment of a sweat measurement system 520, which may be used on its own or as part of any of the method embodiments described above. In this embodiment, the sweat measurement system 520 includes a sweat collection kit 521, which may be provided in a pouch, tray or any other suitable packaging. The kit 521 includes one or more adhesive sweat collection patches 522 (e.g., adhesive gauze pad), one or more alcohol wipes 524 (or other disinfectant skin wipes) for cleaning the skin before applying the patch 522, one or more sweat collection trays 526 and/or sweat collection storage tubes 530, a syringe 528, and three (or alternatively any other number of) sweat test strips 532. The sweat collection tray 526 may be used if sweat will be tested immediately or very soon after collection. The sweat collection tube 530 may be used if the sweat will be tested later, stored and/or transported. In some embodiments, the adhesive patch 522 may be placed into the barrel of the syringe 528, and the plunger of the syringe 528 may be used to squeeze the sweat out of the adhesive patch 522 and into the collection tray 526 or tube 530. The designated end of a test strip 532 may then be placed into the collection tray 526 or tube 530 to collect a sufficient sweat sample on the end of the test strip 532. The system 520 may include any number of test strips 532, where one end of each strip 532 is used to collect the sweat sample, and the other end is inserted into a handheld measurement device 534, which is also part of the system 520. Finally, the system 520 includes a computer application 536 for a smart phone 538, tablet or the like. The handheld measurement device 534, which is described further in the Incorporated References, analyzes the sweat sample and transmits sweat sample data wirelessly to the computer application 536, which may conduct further analysis and may generate further data, such as a rehydration protocol.

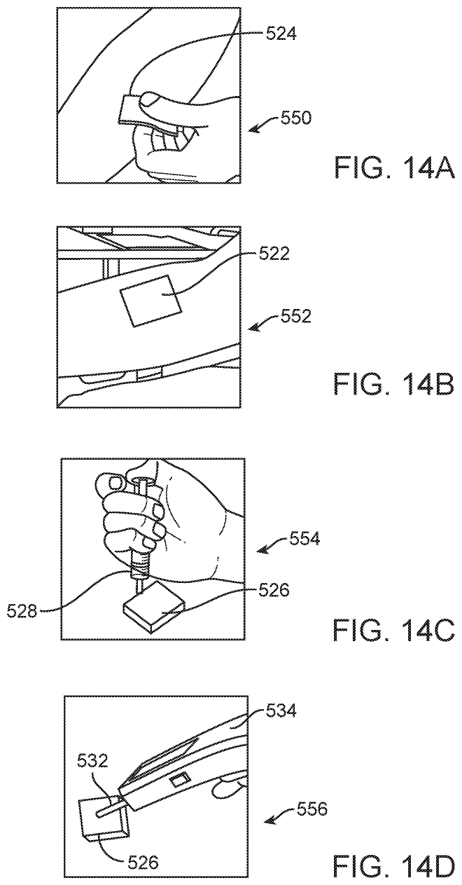

FIGS. 14A-14D illustrate a method for using the system 520 illustrated in FIG. 13 to collect and analyze a sweat sample. As illustrated in FIG. 14A, step 550 involves the test subject (or “user”) wiping down either of her inner forearms with the alcohol wipe 524 from the sweat collection kit 521. Then, as illustrated in FIG. 14B, step 552 involves the user applying the adhesive sweat collection patch 522 to the forearm in the cleaned area. The user will then exercise for a suitable amount of time to collect sweat in the patch 522, for example around 30-60 minutes in one embodiment. (Alternatively, shorter or longer periods of time may be sufficient.) Next, the user removes the sweat collection patch 522, places it in the barrel of the syringe 528, and, as shown in FIG. 14C, step 554, the user depresses the plunger of the syringe 528 to squeeze sweat out of the patch 522 and into the collection tray 526 (or alternatively the collection tube 530). As illustrated in FIG. 14D, the user then places the collection end (or “free end”) of one of the test strips 532 into the sweat sample that resides in the collection tray 526. In this embodiment, the opposite end of the test strip 532 has already been inserted into the handheld measurement device 534, so the user holds the handheld device 534 while inserting the free end of the test strip 532 into the collected sweat sample. Alternatively, the test strip 532 may be inserted into the sweat sample and then into the handheld device 534.

After the measurement device 534 takes a measurement from the first test strip 532, the user removes the first test strip 532, inserts a second test strip 532 into the handheld measurement device 534, inserts the free end of the test strip 532 into the sweat sample, and takes a second measurement. These last steps of the method are repeated for a third test strip 532. In alternative embodiments, fewer than three test strips 532 or more than three test strips 532 may be used for one measurement set. It may be advantageous to use three strips 532, to allow for averaging of three measurements and thus increase accuracy of the test results as compared to using only one or two test strips 532. The handheld measurement device 534 may provide measurements of sweat sodium concentration, sweat osmolarity and/or other sweat characteristics. Measurement data may then be used in any of the methods and algorithms described herein. For example, sweat osmolarity and/or sodium concentration may be used to help the test subject determine how much fluid to consume and what type of fluid (e.g., what quantity and type of electrolytes).

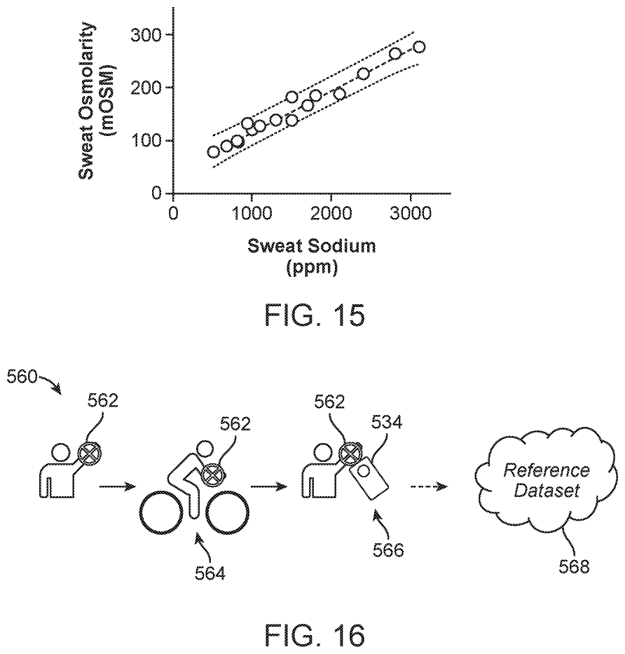

FIG. 15 illustrates the correlation between osmolarity and sodium concentration of various sweat samples. The handheld sweat measurement device 534 wirelessly communicates this result to a phone application for integration into the reference dataset.

Referring now to FIG. 16, another embodiment of a sweat sample collection method 560 is illustrated. In this embodiment, the user first applies a single-use patch 562 to the forearm or other collection area on the skin. The patch 562 in this embodiment includes integrated microfluidics and electrodes. In one embodiment, for example, the single-use patch may include a test strip, such as or the same as the test strips 532 illustrated in FIG. 13. Alternative embodiments may include a different configuration. The user then engages in exercise or other exertion 564, and sweat is collected by the patch 562. For the next step 566, during or after exercise, while still affixed to the test-subject's body, the patch 562 (for example including a test strip 532) is connected to a handheld sweat measurement device 534 to perform measurement(s) of sweat osmolarity, conductivity and/or the like. The handheld device 534 then wirelessly communicates sweat data to a phone application for integration into the reference dataset 568. In some embodiments, the handheld device 534 may also be used to perform other measurements for establishing the reference dataset 568, such as but not limited to saliva osmolarity change and blood lactate concentration.

In another embodiment, a saliva sample is collected directly from the tongue with integrated microfluidics and electrodes. The handheld device 534 wirelessly communicates saliva data to a phone application for integration into the reference dataset. This same system may be capable of performing other measurements for establishing the reference dataset, such as but not limited to saliva osmolarity change and blood lactate concentration.

In another embodiment, a saliva sample is collected by a test subject providing a saliva sample into a receptacle. The sample is then analyzed with integrated microfluidics and electrodes. The handheld device 534 wirelessly communicates saliva data to a phone application for integration into the reference dataset. This same system may be capable of performing other measurements for establishing the reference dataset, such as but not limited to saliva osmolarity change and blood lactate concentration.

Although the above description is believed to be complete and accurate, various changes to any of the embodiments and features described herein may be made, without departing from the scope of the invention.

We claim:

1. A method of measuring an analyte in a bodily fluid sample and combining measurement data from multiple users, the method comprising:
initiating a wireless connection between a handheld analyzer and a smart computing device on which an analyte analysis application has been downloaded;
inserting a test strip into the handheld analyzer;
collecting a sample of a bodily fluid on the test strip;
measuring, with the handheld analyzer, a concentration of at least one analyte in the sample;
wirelessly communicating the measured concentration from the handheld analyzer to the smart computing device;
displaying the measured concentration on the smart computing device;
transmitting the measured concentration to a database; and
organizing data including the measured concentration and at least one additional measured analyte concentration from at least one additional user on the database.

2. The method of claim 1, further comprising initiating a wireless connection between the smart computing device and the Internet, wherein the database is located on a cloud storage location, and wherein transmitting the measured concentration to the database comprises wirelessly transmitting the measured concentration from the smart computing device to the cloud storage location via the Internet.

3. The method of claim 1, wherein the data is organized based upon in groups of multiple users belonging to multiple organizations.

4. The method of claim 1, further comprising initiating the measuring step via the smart computing device, wherein initiating the measuring step comprises:
logging into an operator account on the analyte analysis application;
selecting a specific source from which the sample will be taken; and
confirming the wireless connection between the handheld analyzer and the smart computing device.

5. The method of claim 1, further comprising automatically downloading and storing, on the handheld analyzer, a test strip type and batch data.

6. The method of claim 5, wherein automatically downloading and storing the test strip type and batch data comprises:
measuring a resistance-encoded test strip identification on the test strip; and
comparing the test strip identification with data in a memory of the handheld analyzer to determine the test strip type and batch data.

7. The method of claim 6, further comprising:
communicating the test strip type and batch data to the smart computing device; and
alerting a user through an error message on the handheld analyzer and smart computing device if the test strip type is an unknown test strip type.

8. The method of claim 1, further comprising preventing use of a used or faulty test strip by:
determining that the test strip has already been used or is faulty; and
prompting the user to discard the test strip on at least one of the handheld analyzer or the smart computing device.

9. The method of claim 1, further comprising providing instructions to a user regarding how to collect the sample, using at least one of the application and the handheld analyzer.

10. The method of claim 1, further comprising, using the handheld analyzer:
determining an ambient temperature;
applying a detection technique based on the ambient temperature; and
determining the concentration of the at least one analyte using batch specific calibration coefficients and the ambient temperature.

11. The method of claim 1, further comprising determining, with the handheld analyzer, that a measurement is inaccurate by:
measuring a signal inconsistency; and
detecting an abnormally high signal or an abnormally low signal for the test strip.

12. The method of claim 1, further comprising using the smart computing device to analyze the measured concentration to assist in user interpretation.

13. The method of claim 1, wherein the smart computing device refers a raw measured concentration to a previously established individual specific reference value.

14. A method of measuring at least one analyte in a bodily fluid sample from a subject, the method comprising:
inserting a test strip into a handheld analyzer;
collecting the bodily fluid sample on the test strip by bringing the test strip in contact with a body part of the subject where a bodily fluid is present;
removing the test strip from contact with the body part after the handheld analyzer indicates that a sufficient amount of the bodily fluid sample has been collected;
applying an electrical signal to the test strip;
measuring, with the handheld analyzer, a response of a combination of the test strip and the bodily fluid sample to the applied electrical signal;
analyzing the response with the handheld analyzer to determine that the bodily fluid sample is a valid sample;
measuring a concentration of the at least one analyte in the bodily fluid sample; and
at least one of displaying the measured concentration on the handheld analyzer or transferring the measured concentration to another device to at least one of display the measured concentration, generate further calculations or store the measured concentration.

15. A handheld analyzer for determining a concentration of one or more analytes in a bodily fluid, the handheld analyzer comprising:
a housing;
a test strip port in the housing;
a display screen on the housing;
a temperature sensor in the housing; and
multiple electronic components in the housing, the multiple electronic components comprising:
at least one of a direct digital synthesis (DDS) chip or a digital-to-analog converter (DAC) chip;
an analog-to-digital converter (ADC) chip;
a wireless communication chip;
processing circuitry; and
computer memory.

16. The handheld analyzer of claim 15, wherein the test strip port is configured to accept a test strip selected from the group consisting of analyte specific test strips and test strips capable of measuring multiple analytes.

17. The handheld analyzer of claim 15, wherein the multiple electronic components are configured to automatically transfer test strip configuration settings to the computer memory when the handheld analyzer is connected to a database via a mobile application.

18. The handheld analyzer of claim 15, wherein the handheld analyzer is configured to determine a test strip type and batch data using a resistance-encoded identification on a test strip and data stored in the computer memory.

19. The handheld analyzer of claim 15, wherein the multiple electronic components are configured to automatically adjust a detection method, an excitation waveform and gain settings for multiple types of test strips.

20. The handheld analyzer of claim 15, wherein the temperature sensor is configured to measure an ambient temperature, and wherein the processing circuitry is configured to process the measured ambient temperature using a temperature detection algorithm.
